(19) United States
(12) Patent Application Publication
(10) Pub. No.: US 2014/0107531 A1
(43) Pub. Date: Apr. 17, 2014

(54) INFERENCE OF MENTAL STATE USING SENSORY DATA OBTAINED FROM WEARABLE SENSORS

(71) Applicant: AT&T INTELLECTUAL PROPERTY, I, LP, Atlanta, GA (US)

(72) Inventor: Christopher Baldwin, CRYSTAL LAKE, IL (US)

(73) Assignee: AT&T INTELLECTUAL PROPERTY I, LP, Atlanta, GA (US)

(21) Appl. No.: 13/650,897

(22) Filed: Oct. 12, 2012

(51) Int. Cl. A61B 5/11

(52) U.S. Cl. 600/595

(57) ABSTRACT

Systems and processes that incorporate teachings of the subject disclosure may include, for example, receiving, by a system including a processor, physical states of multiple anatomical locations of a body. Each of the physical states includes one of a position, an orientation, and motion, such as velocity or acceleration, or combinations thereof. A relationship between mental states and body configurations is accessed. A mental state, such as mood or emotion, is determined as the mental state corresponding to a body configuration matching the configuration of a portion of the body corresponding to the physical states of a group of the multiple anatomical locations. Data indicative of the mental state is provided, for example, to adjust another system or application, such as a contextual computer or entertainment system. Other embodiments are disclosed.

### FIELD OF THE DISCLOSURE

The subject disclosure relates to inference of a mental state of an individual using sensory data obtained from sensors worn by the individual.

### BACKGROUND

A current area of technological development receiving attention relates to context computing. Applications of context computing allow various aspects of a given situation to be taken into account when determining a solution. For example, context awareness can be used to link changes in an environment with computer systems operating within the environment. Such contextual awareness can include location awareness, allowing a computing environment to respond to the environment.

### BRIEF DESCRIPTION OF THE DRAWINGS

Reference will now be made to the accompanying drawings, which are not necessarily drawn to scale, and wherein:

FIG. 1 depicts a functional block diagram of an illustrative embodiment of contextual processing system;

FIG. 2 depicts an illustrative embodiment of a process operating in the system described in FIG. 1 and FIGS. 4-7;

FIGS. 3A-3H depict illustrative embodiments of various bodily states determinable by the contextual processing system of FIG. 1;

FIGS. 3I-3J depict illustrative embodiments of an articulating anatomical appendage sensed while in different positions as determinable by the contextual processing system of FIG. 1;

FIGS. 4-5 depict illustrative embodiments of communication systems that provide media services including contextual processing features of FIGS. 1-3;

FIG. 6 depicts an illustrative embodiment of a web portal for interacting with the communication systems of FIGS. 4-5;

FIG. 7 depicts an illustrative embodiment of a communication device; and

FIG. 8 is a diagrammatic representation of a machine in the form of a computer system within which a set of instructions, when executed, may cause the machine to perform any one or more of the methods described herein.

### DETAILED DESCRIPTION

The subject disclosure describes, among other things, illustrative embodiments of techniques for determining a mental state of an individual from sensory data obtained from sensors worn by the individual. A physical state or configuration of at least a portion of a body is determined by an arrangement of wearable sensors. The physical state or body configuration is identified within a relationship between mental states and body configurations, for example, according to an interpreted body language. The mental state, such as mood or feeling is determined as a mental state identified by the relationship. Other embodiments are included in the subject disclosure.

One embodiment of the subject disclosure includes a process including receiving, by a system comprising a processor, physical states of multiple anatomical locations of a body. Each of the physical states includes one of position, orientation, motion, or combinations thereof. The system determines a configuration of a group of the body corresponding to the physical states of a portion of the multiple anatomical locations. A relationship is accessed between a number of mental states and a number of body configurations, and the configuration of the portion of the body is associated with an identified body configuration of the number of body configuration. The system determines a mental state of the number of mental states, such as mood or emotion, corresponding to the identified body configuration, and provides data indicative of the mental state, for example, to adjusting a feature of another system or application, such as a multimedia, advertising, or computer system.

Another embodiment of the subject disclosure includes a system having a memory configured to store computer instructions and a processor coupled to the memory. The processor, responsive to executing the computer instructions, performs operations including receiving sensory data for multiple anatomical locations of a body. The sensory data includes one of position, orientation, motion, or combinations thereof. A physical state of the body is determined from the sensory data. A relationship between a number of mental states and a number of body configurations is received, and the physical state of the body is associated with an identified body configuration of the number of body configurations. A mental state of the number of mental states is determined from the respective physical state of the body, and information indicative of the mental state is generated to control an adjustable feature of another system.

Yet another embodiment of the subject disclosure includes a computer-readable storage medium, including computer instructions which, responsive to being executed by a processor, cause the processor to perform operations including receiving sensory signals from an array of sensors. The sensory signals are indicative of physical states of multiple anatomical locations of a body. Each of the physical states includes one of position, orientation, motion, or combinations thereof. Configuration data is generated corresponding to a configuration of a portion of the body corresponding to the physical states of a group of the multiple anatomical locations. The configuration data is derived from the sensory signals. A relationship between a number of mental states and a number of body configurations is accessed, and the configuration of the portion of the body is associated with a body configuration of a number of body configurations. The configuration data is processed to determine a mental state from the configuration of the portion of the body, and transmission of information is caused over a communication network, wherein the information is indicative of the mental state.

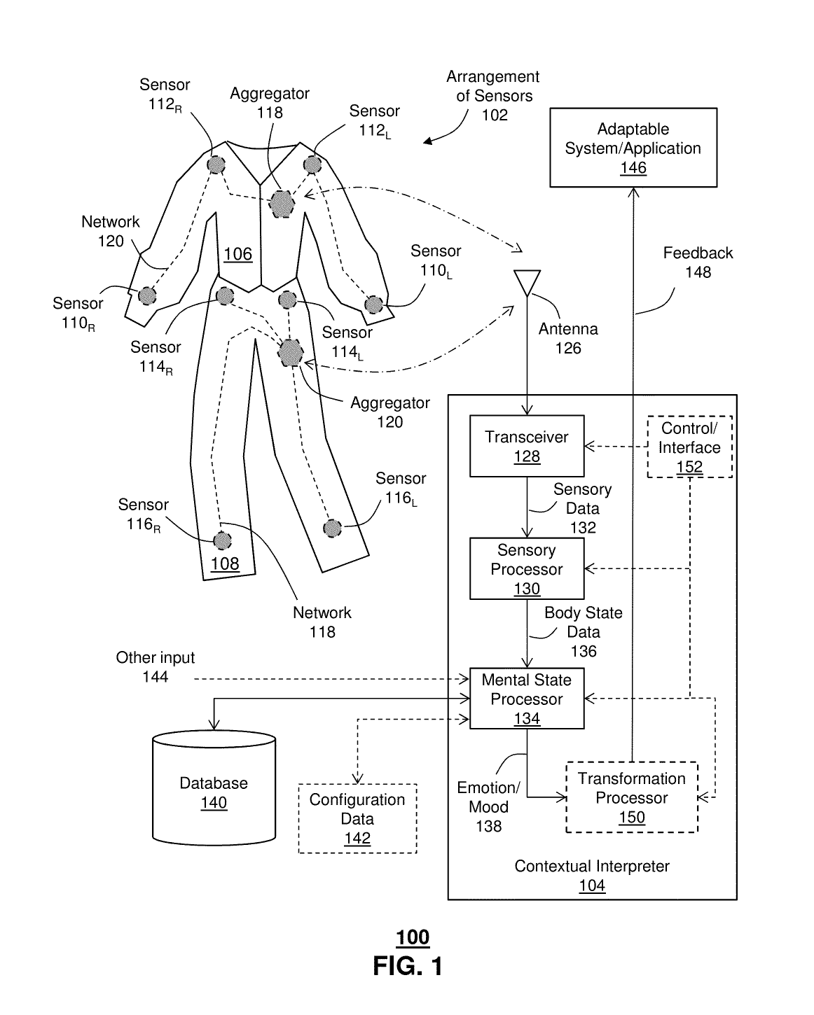

FIG. 1 depicts a functional block diagram of an illustrative embodiment of a contextual processing system 100. The system 100 includes an arrangement of sensors 102 in communication with a contextual interpreter 104. At least a portion of the arrangement of sensors 102 is associated with a wearable article, such as an article of clothing or garment, such as a shirt or blouse 106, a skirt, trousers, or slacks, 108, undergarments, outerwear, and accessories, such as belts, scarves, shoes, glasses, hats, jewelry, watches, rings and the like.

In the illustrative example, a shirt 106 includes left and right wrist or forearm sensors 110_{L}, 110_{R}, and left and right upper arm or shoulder sensors 112_{L}, 112_{R}. Other arrangements of sensors are possible, including fewer or more sensors. For example, the shirt 106 can include one or more sensors at each of the elbows, along a waist or lower section, at a mid section, or along a neck portion. The sensors 110, 112 can be arranged along one or more of front, rear and side portions of the shirt 106. Alternatively or in addition, the trousers 108 are also configured with sensors, including left and right waist or upper thigh sensors 114_{L}, 114_{R} and left and right ankle or lower leg sensors 116_{L}, 116_{R}. Other arrangements of sensors are possible with fewer or more sensors. For example, the trousers can include one or more sensors at each of the knees, along the upper thighs, and at the calves. The sensors can be arranged along one or more of front, rear and side portions of the trousers 108.

Each of the sensors 110, 112, 114, 116 can include one or more sensory elements for sensing or otherwise detecting a physical property and converting it to a signal indicative of the physical property that can be read or otherwise processed. Examples of physical properties, without limitation, include: electromagnetism, such as electric or magnetic fields, light, and heat; and external forces, such as pressure, torque, acceleration or gravity. Conclusions can be drawn from such sensory input as to one or more of a position, angle, displacement, distance, orientation, or movement of the sensor. Movement can include one or more of speed, velocity or acceleration. The sensors can also be configured or otherwise selected to sense proximity or presence. Such physical observations can be repeated for example, according to a schedule, such as periodically, e.g., every few seconds or minutes, or in response to external stimuli, such as motion of the sensor.

The sensors when worn by an individual, gather data relating to one or more physical characteristics, positions, changes, performance, or properties of the individual. This data can be referred to as “biometric” data. In at least some embodiments, biometric data includes biomedical and biomechanical data, and can include any of the following: (i) data tracing a trajectory, speed, acceleration, position, orientation, etc. of one or more of an individual's appendages, torso, head, or other anatomical location; (ii) data reflecting one or more of a heart rate, pulse, blood pressure, temperature, stress level, pH, conductivity, color, blood flow, moisture content, toxin level, viability, respiration rate, etc. of the individual; (iii) data showing whether or not the individual is performing a signal or communication movement (e.g., hand raised, arms crossed, etc.); data showing the posture or other status of the individual (e.g., prone or erect, breathing or not, moving or not); and (iv) data indicative of a mental, or emotional state of the individual.

For example, the sensors can track movement of the subject and/or tension in the subject's muscles. In some embodiments, the sensors can include one or more of the following technologies: (i) accelerometer technology that detects accelerations; (ii) gyroscope technology that detects changes in orientation; (iii) compass or magnetic technology that senses position and/or alignment with relation to magnetic fields; (iv) global positioning system (GPS)-style technology; (v) radio-frequency technology; (vi) proximity sensors including capacitive, inductive and/or magneto-resistive, etc. Sensors can include point sensors sensing biometric information at a location associated with a point or small region of a body.

Alternatively or in addition, sensors can include extended sensors, such as sheet sensors and line sensors. Such extended sensors can include extended elements, such as electrical conductors or fiber optic cables. In operation, movement, distortion, or proximity can impact physical properties detected by such extended sensors. For example, one of a capacitance or an inductance between two extended electrical conductors can vary according to a position of a wearable item to which the extended elements are attached or otherwise incorporated. In particular, such elongated elements can be woven within a fabric of at least some garments.

In some embodiments, multiple sensors are attached to an individual's body, for example, at locations such as those described in Table 1. The anatomical locations and garments/accessories provided in Table 1 are intended to represent illustrative examples and are by no means limiting to either possible anatomical locations of sensors or garments/accessories to which such sensors might be included. Each sensor can include a single sensor or a number of sub-sensor units to determine one or more of the sensed physical properties. In at least some embodiments, the sub-sensor units can include proximity sensors measuring proximity of each sensor to other sensors also worn by the same individual, or to one or more other reference locations that may be located on the individual or at some external location, such as on a communications and/or processing device proximal to the individual.

TABLE 1
Sensor Locations

| Anatomical Location | Garment(s)/Accessory(s) |
|---|---|
| Head | Hats, hoods, earmuffs, earrings |
| Neck | Collars, scarves, necklaces |
| Shoulder (L/R) | Shirts, blouses, sweaters, jackets, vests |
| Upper Arm (L/R) | Shirts, blouses, sweaters, jackets, sleevelets |
| Elbow (L/R) | Shirts, blouses, sweaters, jackets, sleevelets |
| Forearm (L/R) | Shirts, blouses, sweater, jacket, sleevelets |
| Hand (L/R) | Gloves, rings, wristbands, wristwatches |
| Waist | Trousers, skirts, belts |
| Hip (L/R) | Trousers, skirts |
| Upper Thigh (L/R) | Trousers, stockings |
| Knee (L/R) | Trousers, stockings |
| Lower Leg (L/R) | Trousers, legwarmers, stockings, boots |
| Ankle (L/R) | Trousers, legwarmers, stockings, boots |
| Foot (L/R) | Stockings, shoes, sandals, boots, slippers |

In some embodiments, one or more of the shirt sensors 110, 112 are in communication with a first sensory aggregator 118. The first aggregator 118 can also be affixed to, coupled or otherwise embedded within a wearable item, such as the shirt 106, or another wearable item to be worn on the same individual, or located remotely from the individual, for example, at a communications or processing device proximal to the individual. The shirt sensors 110, 112 can be in communication with one or more of other shirt sensors 110, 112 or the first sensory aggregator 118. For example, the shirt sensors 110, 112 and the first sensory aggregator 118 can be communicatively linked by a shirt sensor network 120. The shirt network can include any suitable network for exchanging information, such as sensory signals, identification signals, diagnostic signals, configuration signals, and the like. The shirt sensor network 120 can be hardwired, for example, including electrical conductors and/or optical fibers attached to or otherwise integral to, e.g., woven into, the shirt 106. The network can use any suitable communications protocol, such as universal serial bus (USB), IPX/SPX, X.25, AX.25, proprietary protocols, such as APPLETALK, and TCP/IP. Alternatively or in addition, the shirt sensor network 120 can be a wireless network, using any suitable wireless communications protocol, for example, those compliant with any of the IEEE 802.11 family of standards, such as wireless fidelity, or WiFi, or other personal area networks, e.g., piconet.

In some embodiments, communications can be accomplished in whole or in party using “intra-body communications.” Intra-body communications use a human body or portions thereof to transfer information to or from one or more components, such as sensors, worn on or otherwise proximal to the body. Such intra-body communications can include communications between sensors or with other devices, such as networking, processing or communication devices. Some examples of intra-body communications include “bio-acoustic data transfer,” in which rigid portions of the body, such as bones can be used to transfer information using acoustic transducers. For example, a sensor on an elbow or a knee can include an acoustic transducer to modulate an acoustic signal onto one or more bones of the arm or leg. Other transducers proximal to other joints/bones can detect such transfer of acoustic energy through the body's skeletal system, converting the acoustic energy, for example, into an electrical signal. Other examples of intra-body communications include “electric field data transfer,” in which an electric field can be generated along a surface of the body or within the body, including soft tissues, such as muscle or skin. For example, a sensor adjacent to an anatomical feature can include an electrical transducer generating an electric field within the proximal anatomical feature. Another electrical transducer or circuit element proximal to another anatomical feature of the same body can detect the electric field, converting it, for example, to an electrical current.

In the illustrative example, one or more of the trouser sensors 114, 116 are in communication with a second sensory aggregator 122. The second aggregator 122 can also be affixed to or otherwise embedded within the trousers 108. The trouser sensors 114, 116 can be in communication with one or more of the other trouser sensors 114, 116 or the second sensory aggregator 122. For example, the trouser sensors 114, 116 and the second sensory aggregator 122 can be communicatively liked by a trouser sensor network 124. The trouser sensor network 124 can include any suitable network for exchanging information, such as those described above in relation to the shirt sensor network 120.

It is understood that a single sensory aggregator, such as either of the first or second sensory aggregators 118, 122 can be provided, servicing both the shirt sensors 110, 112 and the trouser sensors 114, 116. In such configurations, the shirt sensor network 120 and the trouser sensor network 124 can be interconnected or otherwise linked allowing for the exchange of information between one or more of the sensors 110, 112, 114, 116 and the aggregator 118, 122. In some embodiments, one or more sensory aggregators are provided on other wearable items, such as in a belt buckle or a wristwatch.

The first and second sensory aggregators 118, 122 can include a communications module for communicating with one or more of the sensors 110, 112, 114, 116, another one of the first and second sensory aggregators 118, 122, or the contextual interpreter 104. The first and second aggregators 118, 122 can include one or more processors executing instructions for processing sensory signals received from one or more of the sensors 110, 112, 114, 116. The sensory signal processing can include one or more of signal conversion, signal interpretation, signal combination or signal aggregation. For example, sensory signal processing can convert electrical sensory signals received from proximity type sensors measuring a capacitance and/or inductance, to one or more of a position, orientation. Such information can be determined using available techniques for locating an object, as in navigation, range finding, triangulation, and the like. Alternatively or in addition processing can be distributed among one or more of the sensors 110, 112, 114, 116, the first and second sensory aggregators 118, 122, the contextual interpreter 104 or any other network accessible processor, for example, available through a telecommunications network or the Internet.

In the illustrative example, each of the first and second sensory aggregators 118, 122 includes a wireless transceiver, such that one or more of the sensors 110, 112, 114, 116 and the first and second aggregators 118, 122 can be communicatively coupled through the aggregator 118, 122 to an external destination, such as the contextual interpreter 104. For applications in which the aggregators 118, 122 aggregate and otherwise process the sensory signals, only the aggregators 118, 122 and not the sensors 110, 112, 114, 116 are in direct communication with the contextual interpreter 104.

In wireless applications, the contextual interpreter 104 includes a wireless transducer, such as an antenna 126, and a transceiver 128 coupled to the antenna 126. The transceiver 134 can be configured to receive sensory data, including aggregated renditions of such data, through the antenna 126, either individually from the sensors 110, 112, 114, 116, or processed by one or more of the sensory aggregators 118, 122. In at least some embodiments, the transceiver 128 can transmit signals to one or more of the sensors 110, 112, 114, 116 or to the sensory aggregators 118, 122, for example to support configuration of the arrangement of sensors 102, transfer software updates, performance of diagnostics, calibrations and the like.

The contextual interpreter 104 also includes a sensory processor 130, a mental state processor 134. The sensory processor 130 receives sensory data 132 from the transceiver 128. The sensory processor 130 processes and otherwise interprets the sensory data 132, including aggregations of such data, to determine one or more states associated with the arrangement of sensors 102 worn by an individual. For example, the states can be states of one or more anatomical locations of a body of the individual, such as the arms, legs, torso, head, feet, hands, and the like. In at least some embodiments, the sensory processor determines a position or positions associated with the anatomical locations of the body, as well as summary interpretations or configurations of larger regions of the body, including substantially the whole body.

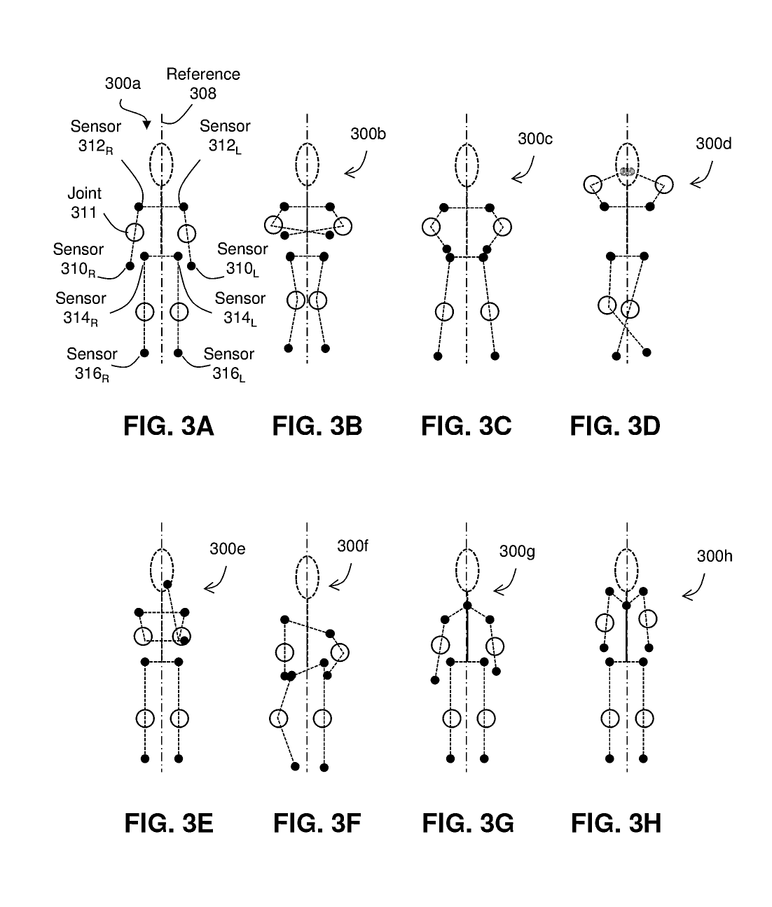

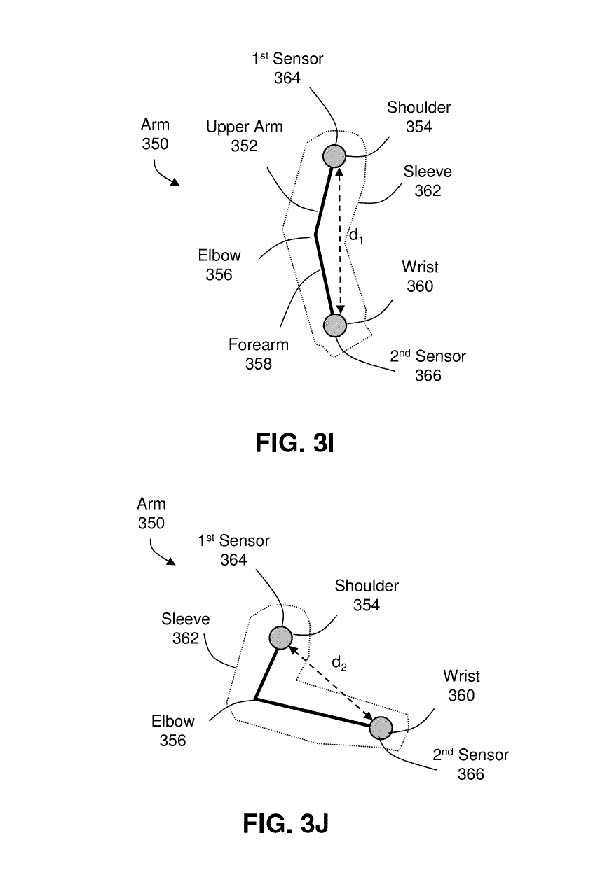

The mental state processor 134 receives the body state data 136 from the sensory processor 130 and processes the body state data 136 to determine a mental state of the individual. The mental state processor 134 is in communication with body language interpretive data, for example, in the form of a body language database 144. It is conceivable that the body language interpretive data could also be in the form of a look up table or other suitable mapping of a body state, such as position, to an inferred mental state, such as emotion or mood. Such databases, interpretive data or reference models can be developed or otherwise constructed from numerous available references on the subject of body language. For example, body state data 136 can identify a body configuration as one of those illustrated in FIG. 3A through 3H. A relationship between such body configurations and corresponding or likely mental state can be provided. In such a list, the body configuration 300d of FIG. 3F might indicate a mood of “relaxed” or a body configuration 300c of FIG. 3E as stressed. Once a body configuration of the relationship is identified by the body state 136, an inference can be drawn that the individual is in the identified mental state or mood.

In some embodiments, the mental state processor 134 is in communication with configuration data 142 (shown in phantom). Configuration data can include information related to the arrangement of sensors 102, for example, associating sensory date originating from particular sensors 110, 112, 114, 116, with an anatomical location proximal to the respective sensor. Alternatively or in addition, configuration data can include information regarding the individual, including one or more of a name or other suitable identifier, as well as physical information, such as age, height, gender, and other self descriptive information such as mannerisms, and the like. Such configuration data can be input or otherwise updated by an individual user, by a system manager, or automatically through information obtained from one or more of the sensors 110, 112, 114, 116 during a configuration exercise. Such configuration exercises might include a scripted set of bodily positions or similar exercises to be undertaken by an individual while wearing the arrangement of sensors. This might include a sequence of fixed positions, e.g., sitting, standing, folded arms, crossed legs, etc., which can be used to interpret and otherwise correlate to sensory data. Such configurations including calibrations and similar settings can be stored within the configuration data either locally to the contextual interpreter 104 or remotely.

In at least some embodiments, the mental state processor 134 receives information from one or more other sources 144. Other sources 144 can include other biometric sensors arranged to collect biometric information from the same individual wearing the arrangement of sensors 102. Such biometric information can include biomedical information, such as heart rate, pulse, perspiration, blood pressure, body temperature, pupil dilation, gaze focus, and the like. Alternatively or in addition, such information from other sources 144 can include information obtained from an external device under the control or direction of the same individual wearing the arrangement of sensors 102. Such information can include, without limitation, responses to user manipulation of a user entry device, such as a keyboard, a mouse, a joystick, of a communication and/or communications device. Such other information 144 can include user profile information as might be obtained from a user's preferences to certain activities, or obtainable from an external source, such as the Internet. For example, a user profile can be assembled according to particulars of multimedia consumed by the user (e.g., song playlists, movie or television watch lists), purchase history (e.g., online purchases), and other demographics, such as age, geographic location, social status, and the like.

The determined emotional state or mood 138 can be provided to an adaptable system or application 146, for example, in the form of feedback 148, such as data indicative of the mental state. The adaptable system or application 146 can use the mental state feedback 150 to adapt the system or application responsive to the individual's inferred mood. Such adaptations can include adjustment of configuration settings of a system, such as a home entertainment system, a home environmental control system, a computer environment, and the like. Such detection and interpretation of body language allows for effortless customization of a user's experience in any system within which they come into contact. Thus, a common arrangement of worn sensors and contextual interpreter can be applied to more than one different system or application.

Such adaptations can be arranged to promote the inferred mental state, providing an adapted environment consistent with the perceived emotional state or mood (e.g., excited, relaxed, focused, humorous, and amorous). Alternatively or in addition, such adaptations can be arranged or otherwise selected to change an inferred emotional state or mood. For example, if an inferred mood is agitated, sad or angry, a system adaptation can be selected that is likely to promote a relaxed, happy, or calm mood.

In some embodiments the contextual interpreter 104 includes a transformation processor 150 (shown in phantom). The transformation processor 150 receives an indication of an inferred mental state or mood 138 from the mental state processor 134 and converts it as may be required for input to the adaptable system or application 146. For example, such conversions might include mapping or similar transformation of mental state to configuration settings suitable for adapting the adaptable system or application 146 in response to the inferred mental state as disclosed herein.

In at least some embodiments, the contextual interpreter 104 includes a control interface 152. The control interface allows for interaction as may be necessary for the configuration, control and operation of the contextual interpreter. For example, configuration data 142 can be entered or otherwise updated through the control interface 152.

The contextual interpreter can be implemented as a standalone device, such as a dedicated computer terminal, or as a subsystem or feature of another device. For example, the contextual interpreter 104 can be implemented in a user mobile device, such as a mobile telephone, tablet computer, personal digital assistant, laptop computer, game console, game console control adapter, personal computer, telephone, media processor, display device and the like. Also, functionality of the contextual interpreter can be distributed across one or more such devices, or remotely located, for example, being accessible through a network, such as the Internet.

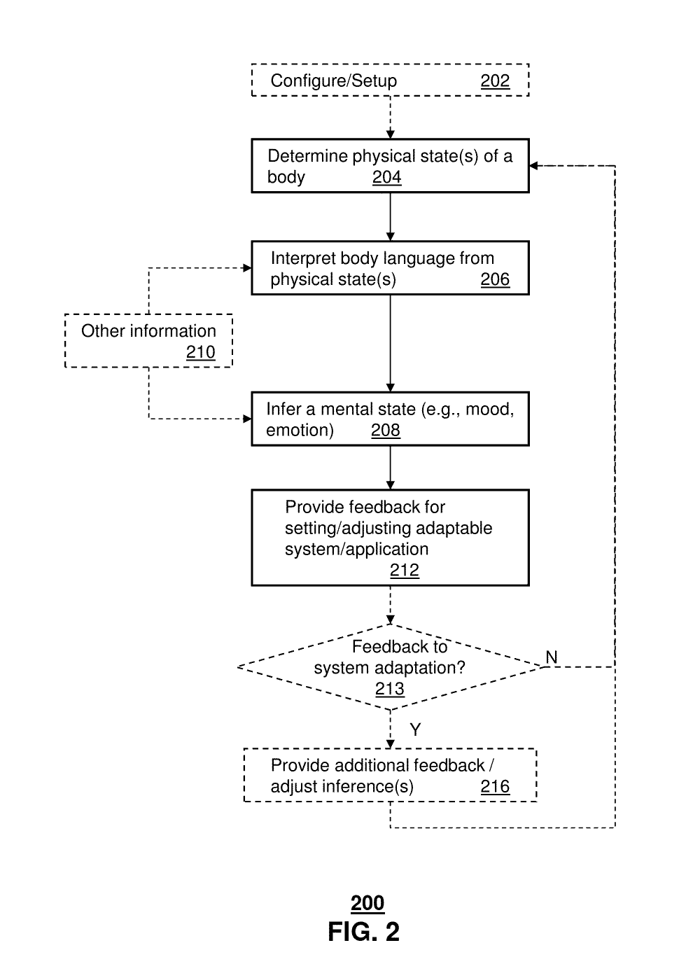

An illustrative embodiment of a process operating in portions of the example systems described herein is provided in FIG. 2. A physical state of a body is determined at 204. The physical state can include a position, orientation, configuration, pose or motion of an entire body, or one or more portions of the body. Such indications of the physical state can be obtained using arrangements of sensors worn on the body, such as those described in FIGS. 1 and 3. For example, sensors can be positioned at predetermined anatomical locations, such as along anatomical appendages or torso, proximal to articulating joints or along anatomical portions between or otherwise spaced apart from such joints. The sensors can use any suitable techniques to determine physical indications of one or more of position, orientation or movement, referred to generally as biomechanical information. Such information can determine one or more of an absolute position of one or more sensors of such arrangements of sensors, or a relative position of groups or subsets of such sensors.

An interpretation of a body language is determined at 206 at least in part from the physical state of the body. For example, physical state information related to position, configuration or orientation of the whole body, or a portion of the body, such as the head, arms or legs, can be used to interpret a message through the principles of reading or otherwise interpreting body language. Examples can include a lookup table, or other such relationship mapping a body configuration to a mental state. A mental state, such as mood, emotion or intention can be inferred at 208 from the interpreted body language.

In at least some embodiments, other information can be provided at 210 (shown in phantom) to one or more of the acts of interpreting body language from the physical state at 206 and inferring a mental state at 208. Other information can include environmental factors, such as date, time, location, lighting, temperature, sounds, video, configuration information, identification of the individual for which a mental state is being inferred, historical information related to the individual, preferences, etc.

Feedback, such as data indicative of a determined mental state is generated at 212 for setting or otherwise adjusting one or more features of an adaptable system or application. Such feedback can be supplied in real time, or near real time to various systems and applications to provide a personalized user experience. As described herein, such systems or applications can relate to telecommunications, for example, by selectively screening calls, selecting voice messages, or ringtones, and the like according to an inferred mental state. Another example related to entertainment systems and applications in which such features as sound level, lighting, color palate for images including video images, menu look, feel and content, including electronic programming guide, recommendations, playlists, video libraries, and the like. Still other examples relate to physical environments, such as home or office environments in which one or more adjustable features, such as lighting or temperature are set or otherwise adjusted according to inferred mental state.

Other systems and applications suitable for using data indicative of a determined mental state relate to advertising. For example, an advertising server receiving a determined mental state of one or more individuals can use the information to increase effectiveness of an advertisement or advertising campaign. For example, an ad server can select one or more advertising messages in response to the determined mental state of an individual, choosing those ads having a greater likelihood of being effective when associated with a particular mental state. Such ads might be for energy boosting products for an individual perceived to be in a sleepy or sluggish mental state. Other ads might be targeted to physical state of the individual, for example, if the individual is exercising, reclining, etc.

In some embodiments, an ad server has access to a repository of advertising or commercial messages. The ad server also has access to associations, such as a lookup table, between advertising or commercial messages of the repository and one or more mental states. Such associations can include positive associations indicating mental states likely to promote receptiveness of the ad/commercial message. Alternatively or in addition, such associations can include negative associations indicating mental states likely to inhibit receptiveness of the ad/commercial message. Thus, the ad server can receive mental state information, consult the associations and select an ad/commercial to which the individual or group is likely to be most receptive. For group ads/messages, statistical analyses can be applied to the mental states of the collective group to select ads/messages most likely to have the greatest positive effect for the group.

In some embodiments, the process includes an initialization, setup or configuration at 202. Such initialization or configuration can be used to accept manual entry from a user including such features as height, weight, gender, name, preferences to any of the adjustable environmental features, playlists, video libraries, etc. Alternatively or in addition, such information can be obtained unobtrusively, for example, through additional sensors, such as scales, stress or strain gauges, cameras, and the like.

In some embodiments, one or more of the process of inferring mental state 208 or providing feedback for adapting a system or application 212 can be modified according to user feedback. For example, a presence of feedback can be detected at 213 (shown in phantom). Feedback can include input from a user, for example, received through the control interface 142 of the contextual interpreter 104. The user feedback may request re-adaptation of an adaptable system or application 146 adapted in response to body state data as disclosed herein. Such re-adaptation might be required to adjust an adaptation founded upon an ambiguous mental state determined by the mental state processor 134. For example, substantially the same body state data might be associated with more than one mental state. Accepting user feedback allows for the state to be re-adapted to suit a user's needs or preferences.

In at least some embodiments, responsive to detecting feedback at 213, the additional feedback is obtained and acted upon at 216 (shown in phantom), for example, to re-adapt the adaptable system or application. Alternatively or in addition, the feedback is used to adjust the inferences adopted at 208, for example, by the mental state processor. In such a manner it is possible to train or otherwise refine performance of the contextual interpreter during extended use and exposure to greater number of situations monitored by way of the sensory data. The system can learn unique mannerisms as well as adapt the interpretation or processing of sensory data to better match a particular user. Such training or refinements can be stored or otherwise retained. In some embodiments, such tailoring is associated with a particular user of a group of users. Alternatively or in addition, such tailoring can be applied in a global sense to all users.

It is also understood that in at least some embodiments, determination of the physical state can include other information, such as sensory information from other sensors detecting biometric information, such as blood pressure, heart rate, pulse, perspiration, temperature, muscle tension, brain activity, speech, images, video images, and the like. Useful information might be contained in sensors or tags in each article of clothing or jewelry, such as physical dimensions, materials, colors, styles, etc., which could be used to enhance the system.

FIG. 3A through FIG. 3H depict illustrative embodiments of various bodily states detectable by sensor arrangements 102 of the contextual processing system 100 of FIG. 1. For example, referring to FIG. 3A, an arrangement of sensors 302, includes left and right shoulder sensors 312_{L}, 312_{R}, left and right wrist or forearm sensors 110_{L}, 110_{R}, left and right waist sensors 114_{L}, 114_{R}, and left and right ankle or lower leg sensors 116_{L}, 116_{R}. The positions, whether absolute, relative or some combination of both, can be detected according to the techniques disclosed herein, such that a model 300a or suitable summary description of an individual wearing the arrangement of sensors 302 can be formed or otherwise estimated. Summary descriptions can include identification of a position, orientation or configuration of an anatomical location. For example, a textual or numeric interpretation can include descriptive features such as: whether a body is seated, standing or reclining; whether the arms are raised or lowered; whether the arm are crossed; whether the legs are spread apart or close together; whether the legs are crossed, and the like.

It is understood that in the illustrative examples reflect a two-dimensional depiction of the relative positions of the arrangement of sensors 302 viewed from a front or back of the body. Similar views can be generated for views at other angles, such as left or right sides, etc., or more generally, the model 300a can be formed in three-dimensional space, such that each sensor and/or joint 311 is positioned with respect to three axes (e.g., x, y, z).

Each of the sensors 310, 312, 314, 316 can be associated with a respective anatomical location according to a respective position on the wearable item. Thus, a location of right shoulder sensor 312_{R}, when worn by an individual, is presumed to be representative of a right shoulder of the individual, because the sensor 312_{R} is affixed to a right shoulder portion of a wearable item, such as a shirt 106 (FIG. 1). Dotted lines are shown extending between selected pairs of sensors, reflecting anatomical regions or appendages extending between pairs of sensors (e.g., arms, legs). The dashed lines can be drawn straight, for example, when a location of a pair of sensors, such as the shoulder and forearm sensors 312, 310 is indicative of a straightened joint 311. Referring to FIG. 3B, the dashed lines can be drawn as bent, for example, when locations of a pair of sensors 312, 310 is indicative of a bent joint 311.

It is possible for estimations of relative and/or absolute positions of sensors 310, 312, 314, 316 of the arrangement of sensors 300a to be interpreted as a position of one or more portions of a body. Having determined such bodily positions or orientations, a mental state, such as emotion, mood or intention can be inferred according to principles of body language. For example, a first body position estimated from the first arrangement of sensors 300a of FIG. 3A with limbs extended and straightened might indicate a relatively alert and/or benign mood; whereas, a second body position estimated from the second arrangement of sensors 300b of FIG. 3B with folded or crossed arms, and relatively closed legs might indicate a nervous mood or an indication that the individual is closed to an environmental situation, such as ideas and/or content being presented at that time. An arrangement of sensors 300c shown in FIG. 3C suggests the body is positioned with hands on hips and legs spaced apart. Such positions can be interpreted, for example, as anger or an assertion of authority. Another arrangement of sensors 300d shown in FIG. 3D suggests the body is positioned with hands behind the head, while legs are crossed. Such positions can be interpreted, for example, as relaxed, content, or pleased, for example, with an environmental situation, such as media being presented at that time.

Another arrangement of sensors 300e shown in FIG. 3E might be inferred as interested, pensive, or thoughtful mood; whereas an arrangement of sensors 300f shown in FIG. 3F might be inferred as confidence. Likewise, yet another arrangement of sensors 300g shown in FIG. 3G might be inferred as tired, meek or timid; whereas, an arrangement of sensors 300h shown in FIG. 3H might be indicative of a tense, or unsure mood. Other interpretations of mental states are possible for any of the illustrated positions 300a-300h, as well as any other conceivable position detectable by such an arrangement of sensors 300.

Alternatively or in addition, more than one mental state can be associated with one or more of the various arrangements of sensors 300. Selection of a particular one of such various arrangements can be determined according to other sensory data, such as biomedical sensory input obtained from other sensors, not shown. Thus, a body temperature and/or heart rate might be used to differentiate anger from boldness or brashness of a configuration, such as shown in FIG. 3C. Still other factors, such as environmental conditions, can be considered alone or in combination with the sensory data to improve or otherwise select among several possible interpretations of mental state. For example, if the environment indicates an individual is watching a horror movie, then a configuration 300h might indicate fright; whereas, if the individual is engaged in a conversation (e.g., a telephone or video) conversation, the same configuration might indicate tension or a lack of confidence.

FIG. 3I and FIG. 3J depict illustrative embodiments of an articulating anatomical appendage, an arm 350, sensed while in different positions as determinable by the contextual processing system 100 of FIG. 1. The arm 350 includes an upper arm 352 extending between a shoulder 354 and an elbow 356, and a lower arm or forearm 358 extending between the elbow 356 and a wrist 360. The arm 350 is covered b a long sleeve 362 that might be part of a wearable garment, such as a shirt, sweater, or jacket 106 (FIG. 1). The sleeve 362, when worn by an individual, includes a first sensor 364 proximal to the shoulder 354, and a second sensor 366 proximal to the wrist 360.

With reference to FIG. 3I, a joint formed at the elbow 356 is shown in a somewhat straightened or extended position. When so positioned, the first and second sensors 364, 366 are separated by a first distance d₁. At least some sensors, such as proximity sensors, can be configured to measure a physical property, S₁ such as a capacitance or an inductance between the first and second sensors 364, 366. As shown in FIG. 3J, the elbow 356 is in a somewhat bent position. When so positioned, the first and second sensors 364, 366 are separated by a second distance d₂. The physical property S₂ sensed by the first and second sensors 364, 366 will be different as the sensors are separated by the d₂, which differs from the first distance d₁. Thus, the measured physical properties S₁, S₂ can be used to determine an approximate position or configuration of the elbow. A measurement of S₁ can be calibrated or otherwise estimated as an extended, or substantially straightened arm 350; whereas, the measurement S₂ can be similarly calibrated or estimated as a bent arm 350. For example, a measured physical property S can be compared with one or more ranges of such properties to determine whether the measured property is indicative of a straightened or bent configuration. When combined with other measurements at the same sensors 364, 366 and/or different sensors, additional information can be determined, such as whether the arm 350 is extended outward, or across a torso.

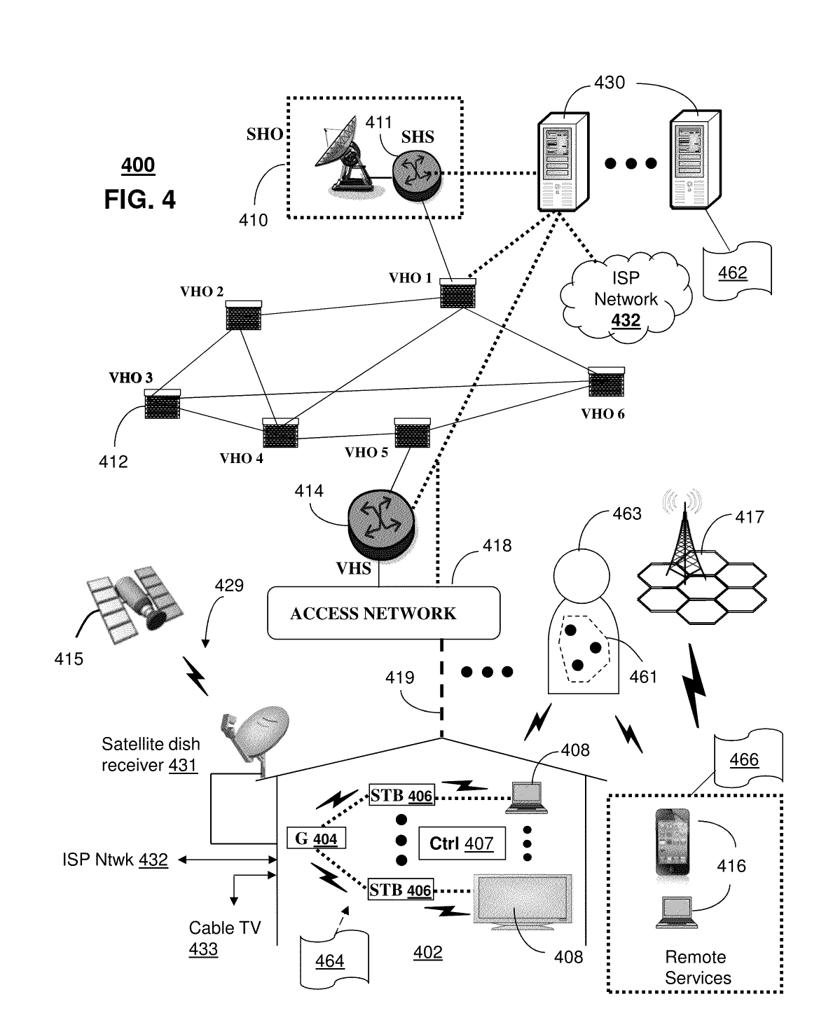

FIG. 4 depicts an illustrative embodiment of a first communication system 400 for delivering media content. The communication system 400 can represent an Internet Protocol Television (IPTV) media system. Features can be provided to interpret sensory information received from an arrangement of sensors 461 worn by an individual 463. The sensory information at least in part describes a physical state of the user, including positions, orientations, or motions of the user's body or portion(s) thereof. A mental state, such as mood or emotion, can be inferred or otherwise detected from the configuration of the user's the body, for example, according to a predetermined relationship between body configurations and mental states. Feedback indicative of the mental state can also be generated and used to control one or more adaptable aspects of a system or application, such as IPTV. In at least some embodiments, the feedback is suitable for adjusting a feature of another system or application, such as a multimedia, advertising, or computer system.

The IPTV media system 400 can include a super head-end office (SHO) 410 with at least one super headend office server (SHS) 411 which receives media content from satellite and/or terrestrial communication systems. In the present context, media content can represent, for example, audio content, moving image content such as 2D or 3D videos, video games, virtual reality content, still image content, and combinations thereof. The SHS server 411 can forward packets associated with the media content to one or more video head-end servers (VHS) 414 via a network of video head-end offices (VHO) 412 according to a multicast communication protocol.

The VHS 414 can distribute multimedia broadcast content via an access network 418 to commercial and/or residential buildings 402 housing a gateway 404 (such as a residential or commercial gateway). The access network 418 can represent a group of digital subscriber line access multiplexers (DSLAMs) located in a central office or a service area interface that provide broadband services over fiber optical links or copper twisted pairs 419 to buildings 402. The gateway 404 can use communication technology to distribute broadcast signals to media processors 406 such as Set-Top Boxes (STBs) which in turn present broadcast channels to media devices 408 such as computers or television sets managed in some instances by a media controller 407 (such as an infrared or RF remote controller).

The gateway 404, the media processors 406, and media devices 408 can utilize tethered communication technologies (such as coaxial, powerline or phone line wiring) or can operate over a wireless access protocol such as Wireless Fidelity (WiFi), Bluetooth, Zigbee, or other present or next generation local or personal area wireless network technologies. By way of these interfaces, unicast communications can also be invoked between the media processors 406 and subsystems of the IPTV media system for services such as video-on-demand (VoD), browsing an electronic programming guide (EPG), or other infrastructure services.

A satellite broadcast television system 429 can be used in the media system of FIG. 4. The satellite broadcast television system can be overlaid, operably coupled with, or replace the IPTV system 400 as another representative embodiment of communication system 400. In this embodiment, signals transmitted by a satellite 415 that include media content can be received by a satellite dish receiver 431 coupled to the building 402. Modulated signals received by the satellite dish receiver 431 can be transferred to the media processors 406 for demodulating, decoding, encoding, and/or distributing broadcast channels to the media devices 408. The media processors 406 can be equipped with a broadband port to an Internet Service Provider (ISP) network 432 to enable interactive services such as VoD and EPG as described above.

In yet another embodiment, an analog or digital cable broadcast distribution system such as cable TV system 433 can be overlaid, operably coupled with, or replace the IPTV system and/or the satellite TV system as another representative embodiment of communication system 400. In this embodiment, the cable TV system 433 can also provide Internet, telephony, and interactive media services.

The subject disclosure can apply to other present or next generation over-the-air and/or landline media content services system.

Some of the network elements of the IPTV media system can be coupled to one or more computing devices 430, a portion of which can operate as a web server for providing web portal services over the ISP network 432 to wireline media devices 408 or wireless communication devices 416

The communication system 400 can also provide for all or a portion of the computing devices 430 to function as a contextual processor (herein referred to as context processor 430). The context processor 430 can use computing and communication technology to perform mental state interpretation function 462, which can include among other things, interpretation of a mental or emotional state of a subscriber 463 wearing an arrangement of sensors 461 configured to measure states of various anatomical regions of the subscriber's body. The media processors 406 and wireless communication devices 416 can be provisioned with software functions 464 and 466, respectively, to utilize the services of context processor 430. For example, the software functions can include one or more of the features disclosed in relation to FIG. 2, or otherwise disclosed herein.

Multiple forms of media services can be offered to media devices over landline technologies such as those described above. Additionally, media services can be offered to media devices by way of a wireless access base station 417 operating according to common wireless access protocols such as Global System for Mobile or GSM, Code Division Multiple Access or CDMA, Time Division Multiple Access or TDMA, Universal Mobile Telecommunications or UMTS, World interoperability for Microwave or WiMAX, Software Defined Radio or SDR, Long Term Evolution or LTE, and so on. Other present and next generation wide area wireless access network technologies can be used in one or more embodiments of the subject disclosure.

FIG. 5 depicts an illustrative embodiment of a communication system 500 employing an IP Multimedia Subsystem (IMS) network architecture to facilitate the combined services of circuit-switched and packet-switched systems. Communication system 500 can be overlaid or operably coupled with communication system 400 as another representative embodiment of communication system 400. One or more features 572 and 576 can be provided to interpret sensory information received from an arrangement of sensors worn by an individual (not shown). The sensory information at least in part describes a physical state of the user, including positions, orientations, or motions of the user's body or portion(s) thereof. A mental state, such as mood or emotion, can be inferred or otherwise detected from the configuration of the user's the body, for example, according to a predetermined relationship between body configurations and mental states. Feedback indicative of the mental state can also be generated and used to control one or more adaptable aspects of a system or application, such as IMS network architecture. In at least some embodiments, the feedback is suitable for adjusting a feature of another system or application, such as a multimedia, advertising, or computer system. Thus, a determined mental state of relaxed can result in a particular selection of entertainment, as in music or movie selections, or program guide selections. Alternatively or in addition, a determined mental state of agitated might also result in a similar selection of soothing or similar environmental stimulus to reduce the individual's agitation. Other examples include selecting environmental stimulus that might be designed to motivate a sleepy or lethargic individual, for example, by increasing lighting, reducing temperature, music selections, and the like.

Communication system 500 can comprise a Home Subscriber Server (HSS) 540, a tElephone NUmber Mapping (ENUM) server 530, and other network elements of an IMS network 550. The IMS network 550 can establish communications between IMS-compliant communication devices (CDs) 501, 502, Public Switched Telephone Network (PSTN) CDs 503, 505, and combinations thereof by way of a Media Gateway Control Function (MGCF) 520 coupled to a PSTN network 560. The MGCF 520 need not be used when a communication session involves IMS CD to IMS CD communications. A communication session involving at least one PSTN CD may utilize the MGCF 520.

IMS CDs 501, 502 can register with the IMS network 550 by contacting a Proxy Call Session Control Function (P-CSCF) which communicates with an interrogating CSCF (I-CSCF), which in turn, communicates with a Serving CSCF (S-CSCF) to register the CDs with the HSS 540. To initiate a communication session between CDs, an originating IMS CD 501 can submit a Session Initiation Protocol (SIP INVITE) message to an originating P-CSCF 504 which communicates with a corresponding originating S-CSCF 506. The originating S-CSCF 506 can submit the SIP INVITE message to one or more application servers (ASs) 517 that can provide a variety of services to IMS subscribers.

For example, the application servers 517 can be used to perform originating call feature treatment functions on the calling party number received by the originating S-CSCF 506 in the SIP INVITE message. Originating treatment functions can include determining whether the calling party number has international calling services, call ID blocking, calling name blocking, 7-digit dialing, and/or is requesting special telephony features (e.g., *72 forward calls, *73 cancel call forwarding, *67 for caller ID blocking, and so on). Based on initial filter criteria (iFCs) in a subscriber profile associated with a CD, one or more application servers may be invoked to provide various call originating feature services.

Additionally, the originating S-CSCF 506 can submit queries to the ENUM system 530 to translate an E.164 telephone number in the SIP INVITE message to a SIP Uniform Resource Identifier (URI) if the terminating communication device is IMS-compliant. The SIP URI can be used by an Interrogating CSCF (I-CSCF) 507 to submit a query to the HSS 540 to identify a terminating S-CSCF 514 associated with a terminating IMS CD such as reference 502. Once identified, the I-CSCF 507 can submit the SIP INVITE message to the terminating S-CSCF 514. The terminating S-CSCF 514 can then identify a terminating P-CSCF 516 associated with the terminating CD 502. The P-CSCF 516 may then signal the CD 502 to establish Voice over Internet Protocol (VoIP) communication services, thereby enabling the calling and called parties to engage in voice and/or data communications. Based on the iFCs in the subscriber profile, one or more application servers may be invoked to provide various call terminating feature services, such as call forwarding, do not disturb, music tones, simultaneous ringing, sequential ringing, etc.

In some instances the aforementioned communication process is symmetrical. Accordingly, the terms “originating” and “terminating” in FIG. 5 may be interchangeable. It is further noted that communication system 500 can be adapted to support video conferencing. In addition, communication system 500 can be adapted to provide the IMS CDs 501, 502 with the multimedia and Internet services of communication system 400 of FIG. 4.

If the terminating communication device is instead a PSTN CD such as CD 503 or CD 505 (in instances where the cellular phone only supports circuit-switched voice communications), the ENUM system 530 can respond with an unsuccessful address resolution which can cause the originating S-CSCF 506 to forward the call to the MGCF 520 via a Breakout Gateway Control Function (BGCF) 519. The MGCF 520 can then initiate the call to the terminating PSTN CD over the PSTN network 560 to enable the calling and called parties to engage in voice and/or data communications.

It is further appreciated that the CDs of FIG. 5 can operate as wireline or wireless devices. For example, the CDs of FIG. 5 can be communicatively coupled to a cellular base station 521, a femtocell, a WiFi router, a Digital Enhanced Cordless Telecommunications (DECT) base unit, or another suitable wireless access unit to establish communications with the IMS network 550 of FIG. 5. The cellular access base station 521 can operate according to common wireless access protocols such as GSM, CDMA, TDMA, UMTS, WiMax, SDR, LTE, and so on. Other present and next generation wireless network technologies can be used by one or more embodiments of the subject disclosure. Accordingly, multiple wireline and wireless communication technologies can be used by the CDs of FIG. 5.

Cellular phones supporting LTE can support packet-switched voice and packet-switched data communications and thus may operate as IMS-compliant mobile devices. In this embodiment, the cellular base station 521 may communicate directly with the IMS network 550 as shown by the arrow connecting the cellular base station 521 and the P-CSCF 516.

It is further understood that alternative forms of a CSCF can operate in a device, system, component, or other form of centralized or distributed hardware and/or software. Indeed, a respective CSCF may be embodied as a respective CSCF system having one or more computers or servers, either centralized or distributed, where each computer or server may be configured to perform or provide, in whole or in part, any method, step, or functionality described herein in accordance with a respective CSCF. Likewise, other functions, servers and computers described herein, including but not limited to, the HSS, the ENUM server, the BGCF, and the MGCF, can be embodied in a respective system having one or more computers or servers, either centralized or distributed, where each computer or server may be configured to perform or provide, in whole or in part, any method, step, or functionality described herein in accordance with a respective function, server, or computer.

The contextual interpreter 430 of FIG. 4 can be operably coupled to the second communication system 500 for purposes similar to those described above. The contextual interpreter 430 can perform function 464 or 466 and thereby provide feedback, based on a body language interpretation of a physical state of a body, for adjusting delivery of services to the CDs 501, 502, 503 and 505 of FIG. 5. CDs 501, 502, 503 and 505, which can be adapted with software to perform function 576 to utilize the services of the contextual interpreter 430. The contextual interpreter 430 can be an integral part of the application server(s) 517 performing function 576, which can be substantially similar to function 466 and adapted to the operations of the IMS network 550.

For illustration purposes only, the terms S-CSCF, P-CSCF, I-CSCF, and so on, can be server devices, but may be referred to in the subject disclosure without the word “server.” It is also understood that any form of a CSCF server can operate in a device, system, component, or other form of centralized or distributed hardware and software. It is further noted that these terms and other terms such as DIAMETER commands are terms can include features, methodologies, and/or fields that may be described in whole or in part by standards bodies such as ^{3rd} Generation Partnership Project (3GPP). It is further noted that some or all embodiments of the subject disclosure may in whole or in part modify, supplement, or otherwise supersede final or proposed standards published and promulgated by 3GPP.

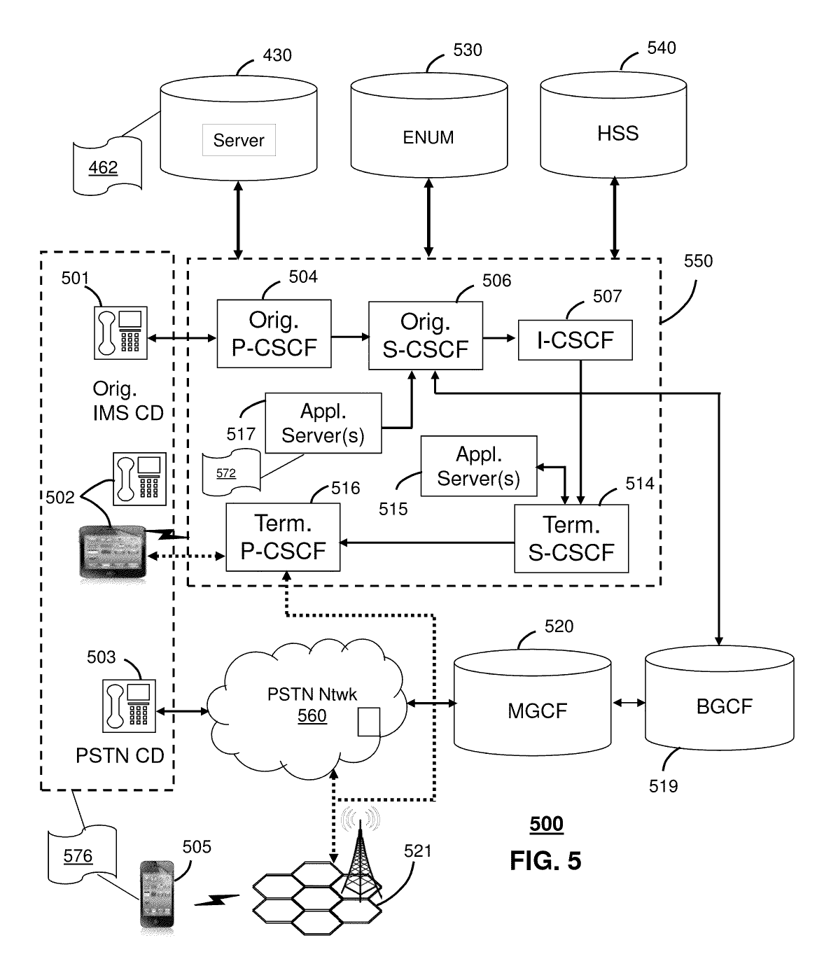

FIG. 6 depicts an illustrative embodiment of a web portal 602 which can be hosted by server applications operating from the computing devices 430 of the communication system 400 illustrated in FIG. 4. Features can be provided to interpret sensory information received from an arrangement of sensors worn by an individual (not shown). The sensory information at least in part describes a physical state of the user, including positions, orientations, or motions of the user's body or portion(s) thereof. A mental state, such as mood or emotion, can be inferred from the configuration of the user's the body. Feedback indicative of the mental state can also be generated and used to control one or more adaptable aspects of a system or application, such the web portal, or services offered through the web portal. In at least some embodiments, the feedback is suitable for adjusting a feature of another system or application, such as a multimedia, advertising, or computer system.

The web portal 602 can be used for managing services of communication systems 400-500. A web page of the web portal 602 can be accessed by a Uniform Resource Locator (URL) with an Internet browser such as Microsoft's Internet Explorer™, Mozilla's Firefox™, Apple's Safari™, or Google's Chrome™ using an Internet-capable communication device such as those described in FIGS. 4-5. The web portal 602 can be configured, for example, to access a media processor 106 and services managed thereby such as a Digital Video Recorder (DVR), a Video on Demand (VoD) catalog, an Electronic Programming Guide (EPG), or a personal catalog (such as personal videos, pictures, audio recordings, etc.) stored at the media processor 106. The web portal 602 can also be used for provisioning IMS services described earlier, provisioning Internet services, provisioning cellular phone services, and so on.

The web portal 602 can further be utilized to manage and provision software applications 464-466, and 572-576 to adapt these applications as may be desired by subscribers and service providers of communication systems 400-500.

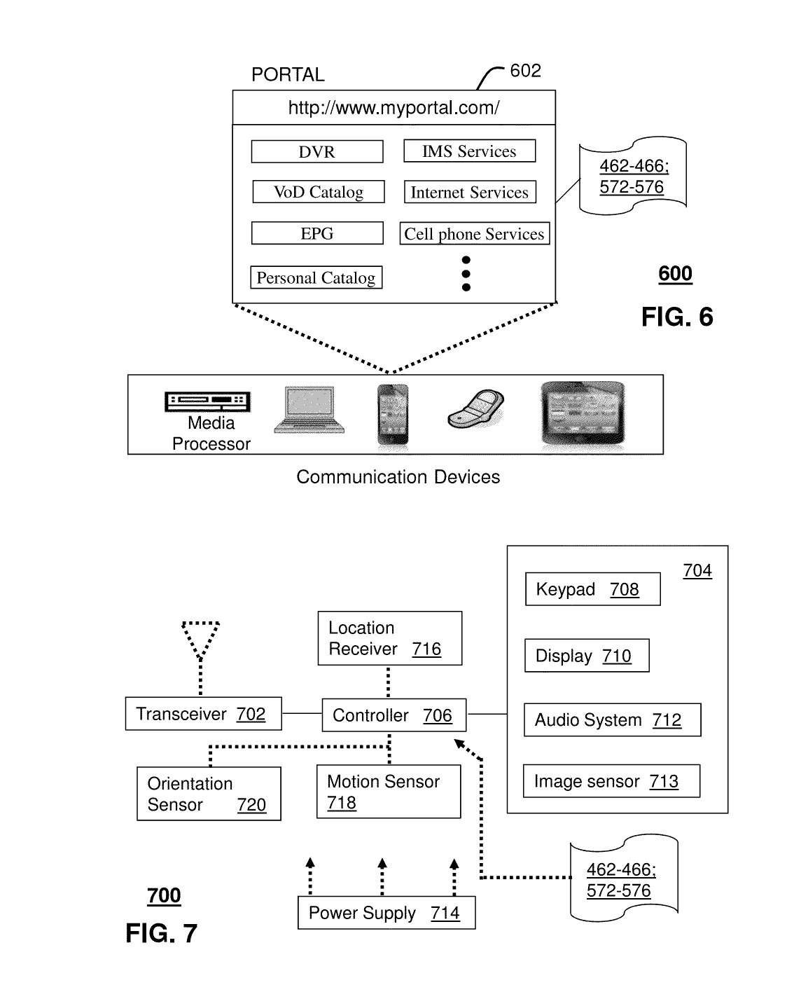

FIG. 7 depicts an illustrative embodiment of a communication device 700. Communication device 700 can serve in whole or in part as an illustrative embodiment of the devices depicted in FIGS. 4-5. Features can be provided to interpret sensory information received from an arrangement of sensors worn by an individual (not shown). The sensory information at least in part describes a physical state of the user, including positions, orientations, or motions of the user's body or portion(s) thereof. A mental state, such as mood or emotion, can be inferred from the configuration of the user's the body. Feedback indicative of the mental state can also be generated and used to control one or more adaptable aspects of a system or application, such the communication device 700, or services accessible through the communication device 700. In at least some embodiments, the feedback is suitable for adjusting a feature of another system or application, such as a multimedia, advertising, or computer system.

The communication device 700 can comprise a wireline and/or wireless transceiver 702 (herein transceiver 702), a user interface (UI) 704, a power supply 714, a location receiver 716, a motion sensor 718, an orientation sensor 720, and a controller 706 for managing operations thereof. The transceiver 702 can support short-range or long-range wireless access technologies such as Bluetooth, ZigBee, WiFi, DECT, or cellular communication technologies, just to mention a few. Cellular technologies can include, for example, CDMA-1X, UMTS/HSDPA, GSM/GPRS, TDMA/EDGE, EV/DO, WiMAX, SDR, LTE, as well as other next generation wireless communication technologies as they arise. The transceiver 702 can also be adapted to support circuit-switched wireline access technologies (such as PSTN), packet-switched wireline access technologies (such as TCP/IP, VoIP, etc.), and combinations thereof.

The UI 704 can include a depressible or touch-sensitive keypad 708 with a navigation mechanism such as a roller ball, a joystick, a mouse, or a navigation disk for manipulating operations of the communication device 700. The keypad 708 can be an integral part of a housing assembly of the communication device 700 or an independent device operably coupled thereto by a tethered wireline interface (such as a USB cable) or a wireless interface supporting for example Bluetooth. The keypad 708 can represent a numeric keypad commonly used by phones, and/or a QWERTY keypad with alphanumeric keys. The UI 704 can further include a display 710 such as monochrome or color LCD (Liquid Crystal Display), OLED (Organic Light Emitting Diode) or other suitable display technology for conveying images to an end user of the communication device 700. In an embodiment where the display 710 is touch-sensitive, a portion or all of the keypad 708 can be presented by way of the display 710 with navigation features.

The display 710 can use touch screen technology to also serve as a user interface for detecting user input. As a touch screen display, the communication device 700 can be adapted to present a user interface with graphical user interface (GUI) elements that can be selected by a user with a touch of a finger. The touch screen display 710 can be equipped with capacitive, resistive or other forms of sensing technology to detect how much surface area of a user's finger has been placed on a portion of the touch screen display. This sensing information can be used to control the manipulation of the GUI elements or other functions of the user interface. The display 710 can be an integral part of the housing assembly of the communication device 400 or an independent device communicatively coupled thereto by a tethered wireline interface (such as a cable) or a wireless interface.

The UI 704 can also include an audio system 712 that utilizes audio technology for conveying low volume audio (such as audio heard in proximity of a human ear) and high volume audio (such as speakerphone for hands free operation). The audio system 712 can further include a microphone for receiving audible signals of an end user. The audio system 712 can also be used for voice recognition applications. The UI 704 can further include an image sensor 713 such as a charged coupled device (CCD) camera for capturing still or moving images.

The power supply 714 can utilize common power management technologies such as replaceable and rechargeable batteries, supply regulation technologies, and/or charging system technologies for supplying energy to the components of the communication device 700 to facilitate long-range or short-range portable applications. Alternatively, or in combination, the charging system can utilize external power sources such as DC power supplied over a physical interface such as a USB port or other suitable tethering technologies.

The location receiver 716 can utilize location technology such as a global positioning system (GPS) receiver capable of assisted GPS for identifying a location of the communication device 700 based on signals generated by a constellation of GPS satellites, which can be used for facilitating location services such as navigation. The motion sensor 718 can utilize motion sensing technology such as an accelerometer, a gyroscope, or other suitable motion sensing technology to detect motion of the communication device 700 in three-dimensional space. The orientation sensor 720 can utilize orientation sensing technology such as a magnetometer to detect the orientation of the communication device 700 (north, south, west, and east, as well as combined orientations in degrees, minutes, or other suitable orientation metrics).

The communication device 700 can use the transceiver 702 to also determine a proximity to a cellular, WiFi, Bluetooth, or other wireless access points by sensing techniques such as utilizing a received signal strength indicator (RSSI) and/or signal time of arrival (TOA) or time of flight (TOF) measurements. The controller 706 can utilize computing technologies such as a microprocessor, a digital signal processor (DSP), programmable gate arrays, application specific integrated circuits, and/or a video processor with associated storage memory such as Flash, ROM, RAM, SRAM, DRAM or other storage technologies for executing computer instructions, controlling, and processing data supplied by the aforementioned components of the communication device 400.

Other components not shown in FIG. 7 can be used in one or more embodiments of the subject disclosure. For instance, the communication device 700 can include a reset button (not shown). The reset button can be used to reset the controller 706 of the communication device 700. In yet another embodiment, the communication device 700 can also include a factory default setting button positioned, for example, below a small hole in a housing assembly of the communication device 700 to force the communication device 700 to re-establish factory settings. In this embodiment, a user can use a protruding object such as a pen or paper clip tip to reach into the hole and depress the default setting button. The communication device 400 can also include a slot for adding or removing an identity module such as a Subscriber Identity Module (SIM) card. SIM cards can be used for identifying subscriber services, executing programs, storing subscriber data, and so forth.

The communication device 700 as described herein can operate with more or less of the circuit components shown in FIG. 7. These variant embodiments can be used in one or more embodiments of the subject disclosure.

The communication device 700 can be adapted to perform the functions of the media processor 406, the media devices 408, or the portable communication devices 416 of FIG. 4, as well as the IMS CDs 501-502 and PSTN CDs 503-505 of FIG. 5. It will be appreciated that the communication device 700 can also represent other devices that can operate in communication systems 400-500 of FIGS. 4-5 such as a gaming console and a media player.

The communication device 700 shown in FIG. 7 or portions thereof can serve as a representation of one or more of the devices of communication systems 400-500. In addition, the controller 706 can be adapted in various embodiments to perform the functions 464-466 and 572-576, respectively.

Upon reviewing the aforementioned embodiments, it would be evident to an artisan with ordinary skill in the art that said embodiments can be modified, reduced, or enhanced without departing from the scope of the claims described below. For example, feedback from the contextual interpreter can be used to tailor an ad campaign not only to a particular individual, but to an inferred mental state of the individual. Other embodiments can be used in the subject disclosure.

It should be understood that devices described in the exemplary embodiments can be in communication with each other via various wireless and/or wired methodologies. The methodologies can be links that are described as coupled, connected and so forth, which can include unidirectional and/or bidirectional communication over wireless paths and/or wired paths that utilize one or more of various protocols or methodologies, where the coupling and/or connection can be direct (e.g., no intervening processing device) and/or indirect (e.g., an intermediary processing device such as a router).

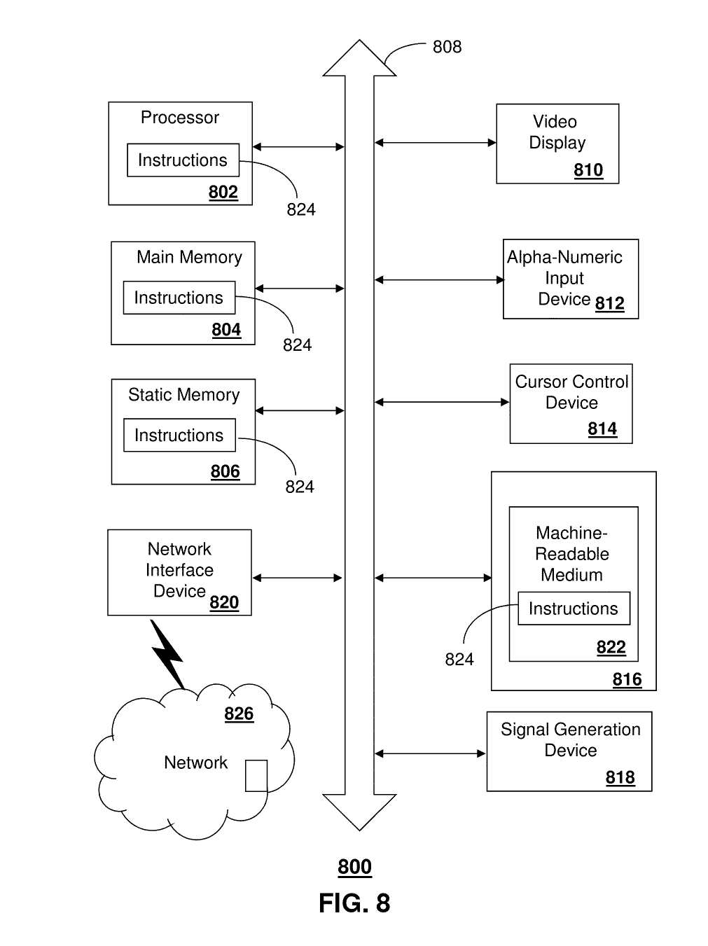

FIG. 8 depicts an exemplary diagrammatic representation of a machine in the form of a computer system 800 within which a set of instructions, when executed, may cause the machine to perform any one or more of the methods describe above. One or more instances of the machine can operate, for example, as the sensors 110, 112, 114, 116, 310, 312, 314, 316, the sensory aggregators 118, 120, the contextual interpreter 104, 430, the transceiver 128, the sensory processor 130, the mental state processor 134, the transformation processor 150, the control interface 152, the adaptable system 146, media processor 406. In some embodiments, the machine may be connected (e.g., using a network 826) to other machines. In a networked deployment, the machine may operate in the capacity of a server or a client user machine in server-client user network environment, or as a peer machine in a peer-to-peer (or distributed) network environment.

The machine may comprise a server computer, a client user computer, a personal computer (PC), a tablet PC, a smart phone, a laptop computer, a desktop computer, a control system, a network router, switch or bridge, or any machine capable of executing a set of instructions (sequential or otherwise) that specify actions to be taken by that machine. It will be understood that a communication device of the subject disclosure includes broadly any electronic device that provides voice, video or data communication. Further, while a single machine is illustrated, the term “machine” shall also be taken to include any collection of machines that individually or jointly execute a set (or multiple sets) of instructions to perform any one or more of the methods discussed herein.

The computer system 800 may include a processor (or controller) 802 (e.g., a central processing unit (CPU), a graphics processing unit (GPU, or both), a main memory 804 and a static memory 806, which communicate with each other via a bus 808. The computer system 800 may further include a display unit 810 (e.g., a liquid crystal display (LCD), a flat panel, or a solid state display. The computer system 800 may include an input device 812 (e.g., a keyboard), a cursor control device 814 (e.g., a mouse), a disk drive unit 816, a signal generation device 818 (e.g., a speaker or remote control) and a network interface device 820. In distributed environments, the embodiments described in the subject disclosure can be adapted to utilize multiple display units 810 controlled by two or more computer systems 800. In this configuration, presentations described by the subject disclosure may in part be shown in a first of the display units 810, while the remaining portion is presented in a second of the display units 810.

The disk drive unit 816 may include a tangible computer-readable storage medium 822 on which is stored one or more sets of instructions (e.g., software 824) embodying any one or more of the methods or functions described herein, including those methods illustrated above. The instructions 824 may also reside, completely or at least partially, within the main memory 804, the static memory 806, and/or within the processor 802 during execution thereof by the computer system 800. The main memory 804 and the processor 802 also may constitute tangible computer-readable storage media.

Dedicated hardware implementations including, but not limited to, application specific integrated circuits, programmable logic arrays and other hardware devices that can likewise be constructed to implement the methods described herein. Application specific integrated circuits and programmable logic array can use downloadable instructions for executing state machines and/or circuit configurations to implement embodiments of the subject disclosure. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the subject disclosure, the processes described herein are intended for operation as software programs running on a computer processor or other forms of instructions manifested as a state machine implemented with logic components in an application specific integrated circuit or field programmable array. Furthermore, software implementations can include, but not limited to, distributed processing or component/object distributed processing, parallel processing, or virtual machine processing can also be constructed to implement the methods described herein. It is further noted that a computing device such as a processor, a controller, a state machine or other suitable device for executing instructions to perform operations on a controllable device may perform such operations on the controllable device directly or indirectly by way of an intermediate device directed by the computing device.

While the tangible computer-readable storage medium 622 is shown in an example embodiment to be a single medium, the term “tangible computer-readable storage medium” should be taken to include a single medium or multiple media (e.g., a centralized or distributed database, and/or associated caches and servers) that store the one or more sets of instructions. The term “tangible computer-readable storage medium” shall also be taken to include any non-transitory medium that is capable of storing or encoding a set of instructions for execution by the machine and that cause the machine to perform any one or more of the methods of the subject disclosure.

The term “tangible computer-readable storage medium” shall accordingly be taken to include, but not be limited to: solid-state memories such as a memory card or other package that houses one or more read-only (non-volatile) memories, random access memories, or other re-writable (volatile) memories, a magneto-optical or optical medium such as a disk or tape, or other tangible media which can be used to store information. Accordingly, the disclosure is considered to include any one or more of a tangible computer-readable storage medium, as listed herein and including art-recognized equivalents and successor media, in which the software implementations herein are stored.

Although the present specification describes components and functions implemented in the embodiments with reference to particular standards and protocols, the disclosure is not limited to such standards and protocols. Each of the standards for Internet and other packet switched network transmission (e.g., TCP/IP, UDP/IP, HTML, HTTP) represent examples of the state of the art. Such standards are from time-to-time superseded by faster or more efficient equivalents having essentially the same functions. Wireless standards for device detection (e.g., RFID), short-range communications (e.g., Bluetooth, WiFi, Zigbee), and long-range communications (e.g., WiMAX, GSM, CDMA, LTE) can be used by computer system 800.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, can be used in the subject disclosure.

The Abstract of the Disclosure is provided with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A method comprising:
receiving, by a system comprising a processor, physical states of a plurality of anatomical locations of a body, wherein each of the physical states comprises one of position, orientation motion, or combinations thereof;
determining, by the system, a configuration of a portion of the body corresponding to the physical states of a group of the plurality of anatomical locations;
accessing, by the system, a relationship between a plurality of mental states and a plurality of body configurations;
associating, by the system, the configuration of the portion of the body with an identified body configuration of the plurality of body configurations;
determining, by the system, a mental state of the plurality of mental states corresponding to the identified body configuration; and
providing, by the system, data indicative of the mental state.

2. The method of claim 1, wherein the physical states of the plurality of anatomical locations are obtained by way of an arrangement of sensors coupled to a wearable item.

3. The method of claim 2, wherein the arrangement of sensors comprises sensors selected from the group consisting of: an accelerometer; a magnetometer; a gyroscope; a capacitive sensor, an inductive sensor; a resistive sensor; and combinations thereof, and wherein each sensor of the arrangement of sensors is selected from the group consisting of: a point sensor; and a linear sensor.

4. The method of claim 1, wherein determining the mental state comprises interpreting the configuration of the portion of the body according to principles of body language.

5. The method of claim 1, wherein the mental state is selected from the group consisting of: emotion; mood; state of mind; frame of mind; intention; feeling; desire; temperament; and combinations thereof.

6. The method of claim 1, wherein determining the configuration of the portion of the body comprises estimating a position of an articulating anatomical appendage from the physical states of a portion of the plurality of anatomical locations disposed on opposite sides of a joint of the articulating anatomical appendage.

7. The method of claim 1, further comprising generating an input control a feature of another system responsive to the data indicative of the mental state.

8. A system comprising:
a memory to store computer instructions; and
a processor coupled to the memory, wherein the processor, responsive to executing the computer instructions, performs operations comprising:
receiving sensory data for a plurality of anatomical locations of a body, wherein the sensory data comprises one of position, orientation, motion, or combinations thereof;
determining from the sensory data a physical state of the body;
receiving a relationship between a plurality of mental states and a plurality of body configurations;
associating the physical state of the body with an identified body configuration of the plurality of body configurations;
determining a mental state of the plurality of mental states from the physical state of the body; and
generating information indicative of the mental state to control an adjustable feature of another system.

9. The system of claim 8, further wherein the sensory data is received from an array of sensors coupled to a wearable article, wherein the processor further performs operations comprising coordinating communication of the sensory data from the array of sensors.

10. The system of claim 9, wherein sensors of the array of sensors are selected from the group of sensors consisting of: an accelerometer; a magnetometer; a gyroscope; a capacitive sensor; an inductive sensor; a resistive sensor; a biometric sensor; and combinations thereof, and wherein each sensor of the array of sensors can be selected from the group consisting of: a point sensor, and a linear sensor.

11. The system of claim 8, wherein the adjustable feature of the other system comprises an environmental feature.

12. The system of claim 8, wherein the mental state is selected from the group consisting of: emotion; mood; state of mind; frame of mind; intention; feeling; desire; temperament; and combinations thereof.

13. The system of claim 8, wherein determining the respective physical state for a portion of the plurality of anatomical locations comprises estimating a position of an articulating anatomical appendage from the physical states of a portion of the plurality of anatomical locations disposed on opposite sides of a joint of the articulating anatomical appendage.

14. The system of claim 8, wherein the other system is selected from the group consisting of: a media delivery system; a media presentation system; an advertising system; a computing environment; a lighting system; a climate control system; a transportation system; and combinations thereof.

15. A computer-readable storage medium, comprising computer instructions which, responsive to being executed by a processor, cause the processor to perform operations comprising:
receiving sensory signals from an array of sensors, wherein the sensory signals are indicative of physical states of a plurality of anatomical locations of a body, wherein each of the physical states comprises one of position, orientation, motion, or combinations thereof;
generating configuration data corresponding to a configuration of a portion of the body corresponding to the physical states of a group of the plurality of anatomical locations, wherein the configuration data is derived from the sensory signals;
accessing a relationship between a plurality of mental states and a plurality of body configurations;
associating the configuration of the portion of the body with a body configuration of a plurality of body configurations;
processing the configuration data to determine a mental state from the configuration of the portion of the body; and
causing a transmission of information over a communication network, wherein the information is indicative of the mental state.

16. The computer-readable storage medium of claim 15, wherein the physical states of a plurality of anatomical locations are received from the array of sensors selected from the group of sensors consisting of: an accelerometer; a magnetometer; a gyroscope; a capacitive sensor; an inductive sensor; and combinations thereof, and wherein each sensor of the array of sensors is selected from the group consisting of: a point sensor; and a linear sensor.

17. The computer-readable storage medium of claim 15, wherein processing the configuration data to determine the mental state comprises interpreting the configuration of the portion of the body according to principles of body language.

18. The computer-readable storage medium of claim 15, wherein the mental state is selected from the group consisting of: emotion; mood; state of mind; frame of mind; intention; feeling; desire; temperament; and combinations thereof.

19. The computer-readable storage medium of claim 15, wherein generating the configuration data comprises estimating a position of an articulating anatomical appendage from the physical states of a portion of the plurality of anatomical locations disposed on opposite ends of a joint of the articulating anatomical appendage.

20. The computer-readable storage medium of claim 15, wherein the other system is selected from the group consisting of: a media delivery system; a media presentation system; an advertising system; a computing environment; a lighting system; a climate control system; a transportation system; and combinations thereof.
